(19) United States
(12) Patent Application Publication
(10) Pub. No.: US 2003/0023196 A1
(43) Pub. Date: Jan. 30, 2003

(54) Shrink wrap bandage

(76) Inventor: Jim Liguori, (US)

Correspondence Address: Mr Jim Liguori, 4892 Basswood, Irvine, CA, 92612, US

(21) Appl. No.: 09/911,970

(22) Filed: Jul. 24, 2001

(51) Int. Cl. A61F 13/00

(52) U.S. Cl. 602/41

(57) ABSTRACT

Shrink-wrap bandages and wound coverings, as well as methods of utilizing the same. According to a preferred embodiment, the bandage is comprised of a sheet of polyvinyl chloride (PVC) or polyolifin shrink film, that is preferably 50 gauge or higher. The sheet is cut to a desired dimension and specifically formulated to undergo a controlled degree of shrinkage upon application of an elevated temperature. According to the preferred embodiment, the PVC is configured to undergo 12-14% shrinkage when subjected to temperatures of 115° Fahrenheit or higher for approximately two to three seconds. The sheet may further preferably include a plurality of apertures formed thereon to allow air to access the covered wound, and may further include an absorbent pad and one or more layers of adhesive to facilitate contact with the skin.

### CROSS-REFERENCE TO RELATED APPLICATIONS (Not Applicable)

### STATEMENT RE: FEDERALLY SPONSORED RESEARCH/DEVELOPMENT

(Not Applicable)

### BACKGROUND OF THE INVENTION

Adhesive bandages are well-known in the art. Generally, most adhesive bandages comprise a plastic backing having inwardly- and outwardly-facing surfaces. Disposed upon the inwardly-facing surface is typically a layer or patch of absorbent material designed to compress against the open wound or sore. Such bandages further typically include a layer of adhesive formed adjacent to or about the periphery of such absorbent material to thus enable the bandage to remain in a fixed position relative the wound to thus provide a protective covering thereover. Exemplary of such adhesive bandages include those sold under the well-known brand names Band-Aid® produced by Johnson and Johnson of Skillman, N.J. and Curad® produced by Beiersdorf, Inc. of Wilton, Conn.

Despite the advantages afforded by conventional adhesive bandages, such as the promotion of rapid healing, wound protection, and the like, the same suffer from numerous drawbacks. Firstly, adhesive bandages necessarily require substantial amounts of adhesive spread out over a sufficiently large surface area to thus enable the same to stick and remain in place around the target wound site. Indeed, such adhesive portion of most adhesive bandages currently in use comprises the major component of such bandages and constitutes a significant portion of the cost associated with manufacture of such a bandage. Moreover, the adhesive component requires complex application, special handling and packaging insofar as the same must be covered with protective wax paper liners and the like to thus enable the layers of adhesive to retain their adhesive properties while in their packaged form.

Notwithstanding the extensive use of adhesives, most adhesive bandages currently in use can and do frequently fail to sufficiently adhere about the wound site. As a result, such bandages detract from the healing process. Such inability to adhere to a specific site on a person's body is exceptionally likely if such site is at an area having a high degree of mobility, such as an elbow or a knee which undergoes constant articulation, particularly with respect to small children.

Moreover, such adhesive can and frequently does become exposed, leaving a sticky residue that attracts dirt and bacteria, and is likewise problematic when it comes time to remove the same, particularly with respect to digits (i.e., fingers and toes) that can touch and contact numerous surfaces, as well as are constantly moving. As virtually everyone who has had an adhesive bandage will appreciate, the adhesive can cause substantial pain when removed from a person's body.

In an attempt to address, at least partially, the drawbacks of conventional adhesive bandages and the like, there is disclosed in U.S. Pat. No. 6,221,447, issued to Munn et al. on Apr. 24, 2001, entitled RUBBER PRODUCTS THAT SHRINK DUE TO THE APPLICATION OF ENERGY AND HYPO-ALLERGENIC RUBBERY PRODUCTS MADE FROM POLYBUTADIENE, TRANS-1,4-POLYBUTADIENE, SYNTHETIC ISOPRENE, SYNTHETIC CIS-1,4-POLYISOPRENE AND THE RESPECTIVE DERIVATIVES THEREOF, rubber-type materials for use as a bandage substitute. Such materials are specifically formed to have a first shape that, after the application of a higher temperature, which is in the range of 94-99° Fahrenheit, conforms to a second, shrunken shape. Such rubbery materials are disclosed for specific uses such as condoms, gloves, catheters, and other rubbery items that are desired to be hypo-allergenic.

Although perhaps well-suited for applications such as condoms and catheters, the same are ill-suited for use as bandages. First of all, insofar as such materials have a relatively low transition temperature (i.e., 94-99° Fahrenheit), such materials are exceptionally difficult to apply, let alone able to sufficiently adhere to the surface of the skin, in warmer climates. In this regard, such rubbery materials will transition prior to even being applied in many instances, oftentimes by merely coming into contact with normal bodily temperature. Such low transition temperature range is further problematic from a shipping standpoint insofar as such materials must be constantly kept and stored at temperatures below the transition temperature, or else such material will prematurely transition to its shrunken operative state prior to their application. Additionally disadvantageous is the fact that such rubbery materials are produced via complex production processes, including precise cross-linking and strictly regulated temperature parameters, that make such materials impractical to mass produce.

Accordingly, there is a substantial need in the art for an improved bandage that more easily and readily adheres to the skin about a wound than prior art adhesive bandages. There is further a need in the art for such a bandage that stays adhered to the skin to a much greater degree than prior art bandages, and further expressly dispenses or substantially minimizes the need for adhesive to be utilized therewith. There is yet further a need for such a bandage that is of exceptionally low cost, may be readily fabricated from existing, commercially-available materials, is safe and easy to use, and can be mass produced and shipped in an extremely economical manner.

### BRIEF SUMMARY OF THE INVENTION

The present invention specifically addresses and alleviates the above-identified deficiencies in the art. More specifically, the present invention is directed to a shrink-wrap bandage that is operative to form a neater, tighter and more secure fit about a wound sought to be protected. Such shrink-wrap bandage is further more sanitary, uses little or no adhesive, is water-resistant and far easier and less expensive to manufacture and ship than prior art bandages and the like.

According to the preferred embodiment, the bandage comprises a sheet of polyvinyl chloride (PVC) or polyolifin shrink film that is designed to assume a first expanded configuration, but transition to a second, shrunken configuration when subjected to elevated temperatures that are preferably approximately 120° Fahrenheit or higher. In this regard, the PVC or polyolifin shrink film is specifically formulated to undergo shrinking from about 8-20% when subjected to such elevated temperatures for about two to three seconds. In more highly refined embodiments, the PVC or polyolifin shrink film will undergo approximately 12-14% shrinkage when subjected to at least approximately 115° Fahrenheit and preferably 120° Fahrenheit or higher for approximately two to three seconds. Among such materials suited for such applications include 500-degree shrink-wrap having a gauge of 50 or greater. In a more highly preferred embodiment, the shrink-wrap comprises 75 gauge shrink-wrap.

In further refinements of the present invention, the shrink-wrap bandage may include a plurality of perforations or apertures, as per conventional adhesive bandages, to enable air to access the covered wound sought to be protected, as well as add comfort and prevent perspiration build-up. Such bandage may further include one or more layers of gauze or absorbent material disposed thereon for contacting with and absorbing fluids from such wound, also as per conventional bandages. Such bandage may further include one or more layers of adhesive to increase the ability of the bandage to remain more securely attached about the wound site. As to this latter aspect, however, it is contemplated that substantially minimal amounts of adhesive will be needed, if at all, due to the superior adhesion accomplished via the shrink-wrap properties of the PVC or polyolifin film.

With respect to the methods of the present invention, there is provided the initial step of placing a bandage of the aforementioned variety over the wound sought to be protected wherein the bandage assumes its first expanded configuration. While positioned over such wound, a heat source is applied to such bandage in a degree sufficient to cause the bandage to transition from its first, expanded configuration to its second, shrunken configuration. Such heat source may comprises any of a variety of well-known sources in the art, including, but not limited to, a conventional hair dryer. Additional energizing sources are contemplated, such as light or radiation, which could facilitate transition of the shrink film.

As a consequence, the shrink film undergoes transition from its expanded configuration to its shrunken, operative configuration. During such transition, the bandage will operatively adhere and become fixed to the tissue about the wound, providing a secure, neat, and tight fit thereabout. Such bandage may thereafter be worn as per conventional bandages and simply removed from the skin following use thereof. Due to its elimination or minimal use of adhesive, as well as the use of PVC and polyolifin, such bandage is water-resistant and exceptionally easy and pain-free to remove.

It is therefore an object of the present invention to provide a shrink-wrap bandage that is substantially effective at adhering to the skin and forming a protective covering about a wound than prior art adhesive bandages and the like.

Another object of the present invention is to provide a shrink-wrap bandage that uses little or no adhesive, is easier to apply, and easier and less painful to remove than prior art adhesive bandages and the like.

Another object of the present invention is to provide a shrink-wrap bandage that is easy and simple to manufacture, requires no special packaging, and is substantially less expensive than prior art adhesive bandages and the like.

Still further objects of the present invention are to provide a shrink-wrap bandage that can be selectively sized and dimensioned for use in a wide variety of wound covering applications, need not be manufactured or used under rigorous temperature control, may be manufactured without the need for complex production processes, results in a bandage that is water-resistant, may be readily fabricated from commercially-available materials and may be readily used in place of conventional prior art bandages and the like.

### BRIEF DESCRIPTION OF THE DRAWINGS

These as well as other features of the present invention will become more apparent upon reference to the drawings wherein:

FIG. 1 is a frontal view of a shrink-wrap bandage constructed in accordance to a preferred embodiment of the present invention.

FIG. 2 is a frontal view of the bandage depicted in FIG. 1 further having formed thereon opposed strips of adhesive layers and an absorbent mid-section for contacting with a wound.

FIG. 3 is a rear-view of the shrink-wrap adhesive bandage depicted in FIG. 2.

FIG. 4a is a perspective view of the leg of an individual depicting the shrink-wrap bandage of the present invention placed upon the knee thereof, the shrink-wrap bandage assuming a first expanded configuration.

FIG. 4b is a perspective view of the leg and bandage of FIG. 4a with a heat source being applied thereto, the latter causing the shrink-wrap bandage to assume a second, operative shrunken configuration.

FIG. 4c depicts the leg and bandage of FIGS. 4a and 4b, wherein the bandage has assumed a most-shrunken configuration.

FIG. 5a is a perspective view of a finger depicting the shrink-wrap bandage of the present invention positioned to be wrapped thereabout.

FIG. 5b is a perspective view of the finger and bandage of FIG. 5a with a heat-source being applied thereto, the latter causing the shrink-wrap bandage to assume a second, operative shrunken configuration.

FIG. 5c depicts the finger and bandage of FIGS. 5a and 5b wherein the bandage has assumed an operative shrunken configuration.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

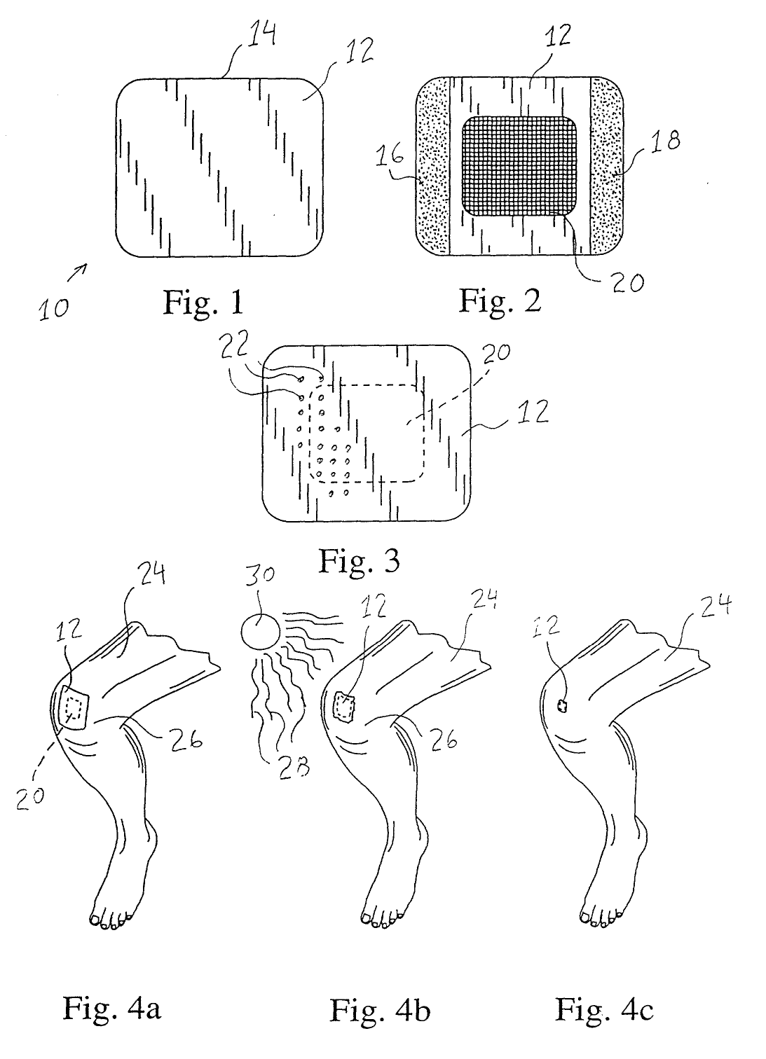

Referring now to the figures, and initially to FIG. 1, there is illustrated a shrink-wrap bandage 10 constructed in accordance to a preferred embodiment of the present invention. As illustrated, such bandage 10 comprises a sheet 12 of thermally-transitional material, namely, shrink film that may be formed and/or cut to have a desired peripheral edge 14. In this regard, it should be recognized at the outset that the bandage 10 of the present invention may be designed to take any of a variety of shapes and configurations as may be desired for applications upon various parts of the human body. Accordingly, the shape of the bandage 10 of the present invention should not be limited to the generally square or rectangular shape as depicted.

The sheet 12 of shrink film preferably comprises either polyvinyl chloride (PVC) or polyolifin shrink film which, as is well-known to those skilled in the art, are well-known and used widely for a variety of product packaging applications. Preferably, sheet 12 will be formed from a film of at least 50 gauge or greater, with 75 gauge shrink film being preferred. It should be understood, however, that shrink films having a lesser gauge may also be possibly utilized in the practice of the present invention.

In an alternative embodiment, the sheet 12 may be coated or laminated on one or both respective sides thereof with a layer of plastic or some other polymer film to insulate such sheet 12. In this regard, to the extent it is undesirable or unwarranted to allow the shrink film to come directly into contact with the user's skin, as discussed more fully below, such laminate or coating may serve as a barrier between the skin and shrink film. Such f ilm or coating may take any of a variety of materials well-known to those skilled in the art, such as polyethylene, polypropylene, and the like.

Such shrink film sheet 12 is operative to undergo a shrinking size transition when subjected to temperatures of approximately 115° Fahrenheit, although temperatures of 120° Fahrenheit or higher may also be deployed. As is wellknown in the art, shrink films, such as PVC and polyolifin shrink films, can be manufactured to precise percentages of shrinking relative their expanded configuration when subjected to specified temperatures. For purposes of practicing the present invention, it is contemplated that any suitable shrink film should be formulated to undergo approximately 8-20% shrinkage when subjected to temperatures of approximately 115° or higher for approximately two seconds or longer. In a more preferred embodiment, such shrink film should undergo approximately 12-14% shrinkage when subjected to approximately 115° for the two to three second time frame. Among such materials suited for such applications include 500-degree shrinkwrap, which is commercially available and well-known in the art, that is formulated to shrink approximately 35% at 500° Fahrenheit. Such material is available from a wide variety of sources, including Reynolds Metals Company of Richmond, Va., and distributed by AJM, Incorporated of San Leandro, Calif.

As discussed more fully below, key to the bandages 10 of the present invention are the ability of the same to undergo such shape transition at such temperature (i.e., approximately 115° Fahrenheit) insofar as such temperature is substantially above normal temperatures typically encountered in shipping and the like and thus will prevent the same from prematurely or inadvertently transitioning. In this regard, in light of the fact that the shrinkage process is irreversible, it should be recognized that such transition temperature be engineered to remain at a sufficiently high level, relative to ordinary environmental temperatures, but at the same time not too high so as to cause damage to the skin and surrounding tissues during application, discussed more fully below.

Referring now to FIG. 2, there are shown additional optional elements to facilitate the functionality of the bandage 10 of the present invention. Upon the sheet 12 may be formed one or more layers of adhesive, such as adhesive layers 16 and 18 depicted. Such adhesives may take any of a variety of those known in the art. While the use of adhesive is considered optional in the practice of the present invention, however, such layers of adhesive 16, 18 may nonetheless be provided to ensure secure attachment of the bandage 10 of the present invention about the wound site. To the extent such adhesive layers are utilized, however, it should be recognized that the same need only be applied in minimal amounts relative to conventional adhesive bandages, which consequently results in a bandage that is substantially easier and less costly to produce.

Such bandage 10 may further include one or more layers of gauze or absorbent material, such as 20, which are designed to absorb fluids from the wound and/or provide a cushioning, protective covering directly over the open wound. Such gauze or padding 20 may take any of a variety known in the art, including those typically employed with conventional adhesive bandages.

Referring now to FIG. 3, there is shown the backside view of the bandage depicted in FIG. 2. As per conventional adhesive bandages, the gauze or absorbent material 20 will preferably be centrally disposed upon the sheet or backing 12. Such sheet 12 may further preferably include perforations or apertures 22, as per conventional bandages, to enable air to access the wound, as is desired to promote the healing process.

Referring now to FIGS. 4a-4c, and initially to 4a, there is shown the method by which the bandage of the present invention is deployed. As an example of how such bandage may be deployed, there is depicted a leg 24 showing the bandage placed upon the knee 26 thereof. As depicted, the sheet 12 is applied over the wound (not shown) such that the gauze 20 is compressed thereagainst as per conventional bandages. As will be appreciated, the bandage is applied such that sheet 12 exists in its first expanded configuration.

Thereafter, as shown in FIG. 4b, heat 28, emanating from a heat source 30, is applied to the bandage such that the sheet 12 thereof transitions from its expanded configuration to its shrunken, operative configuration. As discussed above, such heat 28 will preferably be sufficient to cause the sheet 12 of the bandage to transition, and will preferably be able to attain temperatures of at least approximately 115° Fahrenheit. The source 30 from which such heat emanates may take any of a variety of conventional devices, including conventional hand-held hair dryers and the like. It is further contemplated that any source 30 sufficient to impart the necessary energy to the sheet 12 to enable the same to transition from its expanded configuration to its shrunken configuration will be well-suited for the practice of the present invention. Along these lines, it is contemplated that other energy sources, such as UV or infrared radiation, special lighting, or any of a variety of techniques known in the art may be deployed that are operative to cause shrink film sheet 12 to transition in the desirable manner.

Advantageously, as such sheet 12 transitions from its expanded configuration to its shrunken configuration, the same will adhere to the skin about the wound sought to be protected. In fact, such shrinkage ultimately forms a custom fit about the wound that causes the bandage to stay thereon more securely than prior art adhesive bandages. Indeed, even to the extent minimal amounts of adhesive are utilized, as discussed above with respect to FIG. 2, such lesser amounts of adhesive substantially avoid the problems of prior art adhesive bandages where excessive amounts of adhesive are known to attract dirt and bacteria, as well as cause substantial pain when removed from the individual. Of further advantage is the fact that the shrink film from which the bandage, and more particularly the backing sheet 12 thereof, is water-resistant, more durable and resistant to inadvertent removal when contacted with water or moisture.

In a more exaggerated depiction illustrated in FIG. 4c, the sheet 12 of the present invention is shown having undergone extreme shrinkage such that the same undergoes a substantial reduction in size from its first expanded configuration. Although typically not desired, such reduction in size is provided to illustrate the manner by which the bandage of the present invention transitions from its expanded configuration to shrunken configuration, the process by which advantageously causes the bandage of the present invention to adhere to the surface of the skin. In the illustrated example, it is contemplated that such extreme shrinkage can possibly maximize skin adhesion.

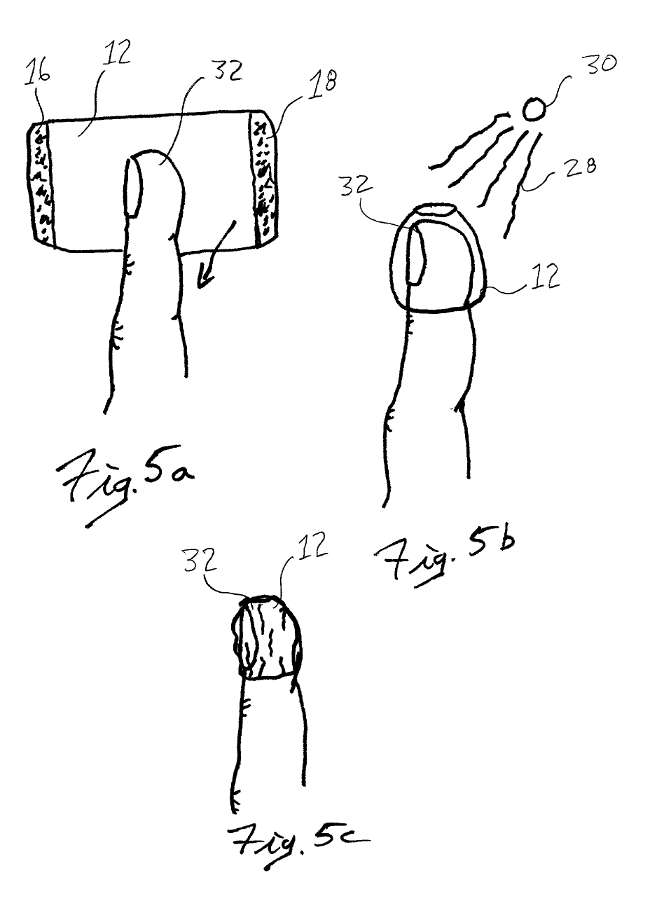

Referring now to FIGS. 5a-5c, initially to FIG. 5a, there is shown a finger 32 positioned to receive sheet 12 thereabout, the latter having two layers of adhesive 16, 18, as accessed above. Preferably, such sheet 12 will be radially wrapped about the finger 32 while in its expanded configuration for subsequent shrinkage. Again, although not required, layers of adhesive 16, 18 maybe provided to facilitate placement of the sheet 12 in the proper position and orientation relative an anatomic structure and in the case depicted a finger 32.

Referring now to FIG. 5b, the sheet 12 positioned about finger 32 is subjected to heat 28 emanating from heat source 30. As discussed above, heat source 30 may take any of a variety of conventional forms, as well as potential energy sources necessary to effectuate the transition of the sheet 12 from its first expanded configurations to its second operative configuration.

After sufficient exposure to the heat 28 from heat source 30, which discussed above will preferably comprise approximately 115° Fahrenheit or higher as applied for approximately two to three seconds, in FIG. 5c the resultant sheet 12 as shrunken about finger 32. While in such configuration, the sheet 12 will tightly adhere radially about the finger 32 for as long as is practical.

As to the removal of the bandage of the present invention, the same may simply be peeled away from the wound site once sufficient duration has passed and/or the bandage needs to be changed. In this respect, such bandage may be easily peeled away with the fingers or tweezers. Advantageously, the bandage does not present any appreciable pain or discomfort when removed therefrom. As is known, prior art adhesive bandages and the like are known to cause substantial pain during their removal.

Additional modifications and improvements of the present invention may also be apparent to those of ordinary skill in the art. For example, although discussed above with respect to PVC and polyolifin, it is contemplated that other shrink films capable of undergoing the desired transition in size when subjected to adequate heat or energy levels may be utilized in the practice of the present invention. Thus, the particular combination of parts and steps described and illustrated herein is intended to represent only certain embodiments of the present invention, and is not intended to serve as limitations of alternative devices and methods within the spirit and scope of the invention.

1. A shrink-wrap bandage for forming a covering about a wound comprising a sheet of shrink film operatively transitional between a first, expanded configuration and a second, shrunken configuration, said bandage being operative to adhere to the skin about said wound as said film transitions from expanded configuration to shrunken configuration.

2. The bandage of claim 1 wherein said shrink film is selected from the group consisting of polyvinyl chloride and polyolifin.

3. The bandage of claim 1 wherein said shrink film is caused to transition from said first, expanded configuration to said second, shrunken configuration when subjected to a temperature of approximately 115° Fahrenheit or higher.

4. The bandage of claim 3 wherein said bandage transitions from said first, expanded configuration to said second, shrunken configuration following at least two seconds of exposure to said temperature.

5. The bandage of claim 1 further comprising:
a) at least one layer of adhesive formed thereon.

6. The bandage of claim 5 wherein said bandage includes at least two strips of adhesive formed thereon.

7. The bandage of claim 1 wherein said bandage further includes an absorbent pad formed thereon.

8. The bandage of claim 1 wherein said bandage further includes a plurality of apertures to provide ventilation to said wound sought to be covered by said bandage.

9. The bandage of claim 2 wherein said shrink film has a gauge of 50 or higher.

10. The bandage of claim 9 wherein said film comprises 75 gauge film.

11. A method for forming a covering over a wound comprising the steps:
a) providing a shrink-wrap bandage, said shrink-wrap bandage being formed from a shrink film operative to transition from a first, expanded configuration to a second, shrunken configuration when subjected to a heat source, said bandage being provided in said first, expanded configuration;
b) placing said bandage in step (a) over said wound; and
c) applying a heat source to said bandage placed over said wound in step (b).

12. The method of claim 11 wherein in step (a), said bandage operatively transitions from its first, expanded configuration to second, shrunken configuration when subjected to a temperature of approximately 115° Fahrenheit or higher; and wherein in step (c), said heat is applied to said bandage at a temperature of approximately 115° Fahrenheit or higher.

13. The method of claim 12 wherein in step (a), said bandage is operative to transition from its expanded configuration to shrunken configuration when subjected to said 115° Fahrenheit or higher temperature for a duration of at least two seconds; and wherein in step (c), said heat is applied for at least two seconds.
